SEQUENCE LISTING (4 sequences)

SEQ ID NO: 1 - DNA, 20 bp, Artificial Sequence
  forward primer for exon 5 of haptoglobin 2
atggctatgtggagcactcg

SEQ ID NO: 2 - DNA, 20 bp, Artificial Sequence
  reverse primer for for exon 7 of haptoglobin 2
tacagggctcttcggtgtct

SEQ ID NO: 3 - DNA, 18 bp, Artificial Sequence
  forward primer for exon 2 of haptoglobin 1 or exon 2 of haptoglobin 2
tttctggctgctaagttg

SEQ ID NO: 4 - DNA, 18 bp, Artificial Sequence
  reverse primer for exon 5 of haptoglobin 1 or exon 7 of haptoglobin 2
aatgtctttcgctgttgc

(19) United States
(12) Patent Application Publication
(10) Pub. No.: US 2014/0363818 A1
(43) Pub. Date: Dec. 11, 2014

(54) EGFR and PAR2 Regulation of Intestinal Permeability

(71) Applicants: Alessio Fasano, Charlestown, MA (US); Karen Manon Lammers, Boston, MA (US); Terez P. Shea-Donohue, Chevy Chase, MD (US); Simeon Glodblum, Baltimore, MD (US); Craig Sturgeon, Severna Park, MD (US)

(72) Inventors: Alessio Fasano, Charlestown, MA (US); Karen Manon Lammers, Boston, MA (US); Terez P. Shea-Donohue, Chevy Chase, MD (US); Simeon Glodblum, Baltimore, MD (US); Craig Sturgeon, Severna Park, MD (US)

(73) Assignee: UNIVERSITY OF MARYLAND, BALTIMORE, Baltimore, MD (US)

(21) Appl. No.: 14/470,389

(22) Filed: Aug. 27, 2014

Related U.S. Application Data
(63) Continuation of application No. 13/323,100, filed on Dec. 12, 2011.
Application No. 13/323,100 is a continuation-in-part of application No. PCT/US2010/000167, filed on Jun. 10, 2010.
(60) Provisional application No. 61/185,662, filed on Jun. 10, 2009.

(51) Int. Cl. C12Q 1/68

(52) U.S. Cl. CPC C12Q 1/6883; C12Q 2600/112; C12Q 2600/158
USPC 435/6.11

(57) ABSTRACT

The present invention provides methods for diagnosing an immune-mediated disease, e.g., an autoimmune disease, an allergy or an inflammatory disease. Diagnosis is made by detecting a heterozygous or homozygous genotype of haptoglobin 2 or by detecting and quantifying pre-haptoglobin 2 mRNA or protein. After diagnosis, the disease may be treated by decreasing cell permeability leading to increased transepithelial electrical resistance, for example, by administering an antibody directed against single chain zonulin thereby inhibiting epidermal growth factor receptor and inhibiting proteinase-activated receptor 2 (PAR₂). Also provided is a single step method for determining severity of an immune-mediated disease in a subject by identifying a genotype for haptoglobin 2 in a biological sample from the subject. A homozygous genotype correlates to 2 copies of zonulin and a more severe disease than a heterozygous genotype correlating to 1 copy of zonulin.

### CROSS-REFERENCE TO RELATED APPLICATION

This application is a continuation under 35 U.S.C. §120 of pending application U.S. Ser. No. 13/323,100, filed Dec. 12, 2011, which is a continuation-in-part under 35 U.S.C. §120 of international application PCT/US2010/00167, filed Jun. 10, 2010, now abandoned, which claims benefit of priority under 35 U.S.C. §119(e) of provisional application U.S. Ser. No. 61/185,662, filed Jun. 10, 2009, now abandoned, the entirety of all of which are hereby incorporated by reference.

### STATEMENT REGARDING FEDERALLY SPONSORED RESEARCH

This invention was made with government support under Grant number DK048373 awarded by the National Institute of Health. The government has certain rights in the invention.

### BACKGROUND OF THE INVENTION

1. Field of the Invention

The present invention relates to the fields of cell biology and intestinal permeability. More specifically, the present invention relates to EGFR and proteinase-activated receptor 2 (PAR₂) regulation of intestinal permeability.

2. Description of the Related Art

Increased hygiene leading to a reduced exposure to various microorganisms has been implicated as a cause for the ‘epidemic’ of allergic, inflammatory, and autoimmune diseases recorded in industrialized countries during the past 3-4 decades (1). Apart from genetic makeup and exposure to environmental triggers, a third key element, i.e., increased intestinal permeability (IP), has been proposed in the pathogenesis of these diseases (2-4).

Intestinal permeability, together with antigen sampling by enterocytes and luminal dendritic cells, regulates molecular trafficking between the intestinal lumen and the submucosa, leading to either tolerance or immunity to non-self antigens (5). However, the dimensions of the paracellular space (10 to 15 Å) suggest that solutes with a molecular radius exceeding 15 Å (˜3.5 kDa) (including proteins) are normally excluded from this uptake route. The intercellular tight junctions (TJs) tightly regulate this paracellular antigen trafficking.

Tight junctions are dynamic structures operative in several key functions of the intestinal epithelium under both physiological and pathological circumstances (3). However, despite major progress in the knowledge on the composition and function of intercellular tight junctions, the mechanism(s) by which they are regulated is(are) still incompletely understood.

The discovery of Vibrio cholerae zonula occludens toxin (Zot), a toxin that increases tight junction permeability, led to the identification of its eukaryotic counterpart, zonulin, as the only physiological mediator known to reversibly regulate intestinal permeability by modulating intercellular tight junctions (6, 7). Human zonulin is a ˜47 kDa protein that increases intestinal permeability in non-human primate intestinal epithelia (7), participates in intestinal innate immunity (8), and is overexpressed in autoimmune disorders in which tight junction dysfunction is central, including celiac disease (CD) (9, 10) and type 1 diabetes (T1D) (11).

Haptoglobin (Hp) is an acute-phase response protein, synthesised mainly in the liver as well as arterial walls, endometrium and peritoneum. The core function of haptoglobin is as a haemoglobin (Hb) binding protein, required for terminal processing and disposal of free haemoglobin, mostly in the reticular endothelial system of the liver. This system allows the iron present in the Hb moiety to be conserved.

Haptoglobin has a tetrameric structure comprising two alpha and two beta chains, linked by disulphide linkages. The β chain (245 amino acids) has a mass of about 40 kDa (of which approximately 30% w/w is carbohydrate) and is shared by all phenotypes. The α chain exists in two forms: α1, (83 amino acids, 9 kDa) and α2 (142 amino acids, 17.3 kDa) and therefore haptoglobin occurs as three phenotypes, referred to as Hp1-1, Hp2-1 and Hp2-2. Hp1-1 contains two α1 chains, Hp2-2 contains two 2 chains, and Hp2-1 contains one α1 and one α2 chain. Hp 1-1 has a molecular mass of 100 kDa, or 165 kDa when complexed with Hb. Hp1-1 exists as a single isoform, and is also referred to as Hp dimer. Hp2-1 has an average molecular mass of 220 kDa and forms linear polymers. Hp2-2 has an average molecular mass of 400 kDa and forms cyclic polymers. Each different polymeric form is a different isoform.

Haptoglobin is a potential treatment for renal disorders caused by haemolysis. It is potentially useful therapeutically as a means of removing free haemoglobin. The complexes thus formed having potential additional benefits as anti-inflammatory, antioxidant or angiogenic agents. However, haptoglobin is considered difficult to isolate in large amounts whilst retaining its biological activity.

There is a recognized need in the art for a functional characterization of pre-haptoglobin 2 as well as methods of regulating intestinal permeability. The present invention fulfills this long standing need in the art.

### SUMMARY OF THE INVENTION

While zonulin's role as an intestinal permeating modulator in health and disease has been described functionally, its biochemical characterization has remained elusive. The present invention shows that through proteomic analysis of human sera, zonulin is identical to pre-haptoglobin (HP)2, a molecule that, to date, has only been regarded as the inactive precursor for HP2, one of the two genetic variants (together with HP1) of human pre-haptoglobins. The present invention demonstrates the functional characterization of zonulin as pre-haptoglobin 2, a multifunctional protein that, in its intact single chain precursor form, appears to regulate intestinal permeability by transactivating the EGFR via PAR₂ activation, while in its cleaved two-chain form acts as a Hb scavenger.

In one embodiment of the present invention, there is provided a method for diagnosing an immune-mediated disease in a subject. The method comprises the steps of obtaining a biological sample from the subject and a healthy control, and, in a single amplification step, performing a genotype amplification of a haptoglobin gene comprising the sample and the control. An increase in copies of a haptoglobin 2 genotype compared to control correlates to a diagnosis and severity of the immune-mediated disease in the subject.

In another embodiment of the present invention, there is provided a method for diagnosing celiac disease. The method comprises amplifying a haptoglobin gene using specific primers in exon 2 and exon 5 of haptoglobulin 1 (HP1) from a biological sample and a healthy control sample. Genotype is determined wherein an increase in copies of a haptoglobin 2 genotype in the biological sample compared to the healthy control sample correlates to a diagnosis and severity of the immune-mediated disease in a subject.

In yet another embodiment of the present invention, there is provided a single step method for determining severity of an immune-mediated disease in a subject. The method comprises identifying a genotype for haptoglobulin 2 (HP2) from an amplified haptoglobin gene in a biological sample from the subject. A homozygous genotype for haptoglobin 2 is indicative of two copies of zonulin gene thereby correlating to a more severe disease than a heterozygous genotype that is indicative of one copy of zonulin.

In yet another embodiment of the present invention, there is provided a method for diagnosing an autoimmune disease in a subject. The method comprises the steps of obtaining a biological sample from the subject and amplifying pre-haptoglobin 2 mRNA in the biological sample. The pre-haptoglobin2 in the amplified product is quantified where an increase in pre-haptoglobin-2 product compared to a control is indicative of the presence of the autoimmune disease.

In yet another embodiment of the present invention, there is provided a method for diagnosing an autoimmune disease in a subject. The method comprises the steps of obtaining a biological sample from the subject detecting pre-haptoglobin 2 protein in the biological sample. The detected pre-haptoglobin 2 protein is quantified where an increased level of pre-haptoglobin-2 in the sample compared to a control is indicative of the presence of the autoimmune disease.

In yet another embodiment of the present invention, there is provided a method of treating an autoimmune disease. The method comprises the step of decreasing cell permeability leading to increased transepithelial electrical resistance. A related method is provided comprising the additional step of inhibiting proteinase-activated receptor 2. Yet another related method is provided comprising the additional step of avoiding zonulin release by gliadin through CXCR3 receptor binding.

In yet another embodiment of the present invention, there is provided a method of treating an autoimmune disease in an individual in need of such treatment. The method comprises the steps of inhibiting epidermal growth factor receptor and inhibiting PAR₂. A related method is provided comprising the additional step of inhibiting gliadin.

In yet another embodiment of the present invention, there is provided a method of treating celiac disease in an individual in need of such treatment. The method comprises the steps of administering an antibody directed against single chain zonulin thereby inhibiting epidermal growth factor receptor and inhibiting proteinase-activated receptor 2 (PAR₂). A related method is provided comprising the additional step of inhibiting gliadin.

In yet another embodiment of the present invention, there is provided a method for diagnosing a disease associated with increased intestinal permeability in a subject. The method comprises the steps of obtaining a biological sample from the subject and measuring an expression level of a pre-haptoglobin or glycoform thereof in the biological sample. The expression level of the pre-haptoglobin or glycoform thereof in the sample is compared with an expression level of the same expressed in a control sample. Overexpression of the pre-haptoglobin or glycoform thereof compared to the control is indicative of the presence of the autoimmune disease.

Other and further aspects, features and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows Western blotting using zonulin cross-reacting anti-Zot polyclonal Ab on CD patient sera that were depleted of albumin and immunoglobulins. Three main patterns were detected: sera showing a 18 kDa immunoreactive band and a fainter ˜45 kDa band (lane 1), sera showing only a 9 kDa band (lane 2), and sera showing both the 18 kDa and 9 kDa bands (lane 3).

FIGS. 2A-2B show Coomassie and Western immunoblotting (WB) of purified human homozygote HP1-1 and HP2-2 both untreated and after deglycosylation with PGNase. FIG. 2A shows that Coomassie staining of untreated HPs showed a shared glycosylated b chain migrating at a MW ˜52 kDa, while the α chain of HP1-1 (α1) and of HP2-2 (α2) migrated at the predicted MW of 8 and 18 kDa, respectively. Deglycosylation with PGNase caused a shift of the β chain to a MW of ˜36 kDa (complete deglycosylation) or higher (incomplete deglycosylation). As expected, no shifts were observed in the non-glycosylated α1 and α2 chains. FIG. 2B is a Western Blot (WB) of purified human homozygote HP1-1 and HP2-2 both untreated and after deglycosylation with PGNase run in triplicate on a single gel, transferred, and then separately subjected to WB analysis using polyclonal anti-Zot (left panel), monoclonal anti-HP (center panel), or polyclonal anti-HP antibody (right panel). The three antibody tested recognized both the α1 and α2 chains (all panels, lanes 1 and 2) whose pattern of reactivity did not change after deglycosylation of both HP1-1 and HP2-2 protein preparations (lanes 3 and 4). Conversely, deglycosylation caused the expected gel mobility shift of the β chain in both HP1-1 and HP2-2 detected by either the anti-HP monoclonal (center panel, lanes 3 and 4) or anti-HP polyclonal antibody (right panel, lanes 3 and 4). The zonulin cross-reacting anti-Zot antibody recognized an extra ˜45 kDa band in HP2-2 but not in HP1-1 that did not shift after deglycosylation (arrows). MS/MS analysis and N-terminal sequencing identified this ˜47 kDa band as pre-HP2.

FIG. 3 shows that zonulin increased intestinal permeability in C57BL/6 WT mice in a dose-and time-dependent manner. Zonulin was applied to the luminal side of C57BL/6 WT intestinal segments at 5, 10, 25 and 50 μg/well. Trypsin-cleaved pre-HP2 was applied at 50 μg/well. Starting at 60 min post-exposure, zonulin induced significant drop in TEER when applied at concentrations 10 μg/well (P value ranging from 0.03 to 0.036). Data are mean values±SEM from 4 independent experiments.

FIGS. 4A-4D show the effect of zonulin on mouse gastrointestinal permeability in vivo. Zonulin (closed bars) (170 mg/mouse) increases both mouse small intestinal (FIG. 4A) and gastroduodenal (FIG. 4B) permeability as compared to BSA-treated controls (open bars). The differences in lacman ratio (small IP) and sucrose fractional excretion (gastroduodenal permeability) are shown as percentage of change in permeability between the measurements on the challenge day and 3 days before challenge. Mature two-chain HP2 (dotted bars) (170 mg/mouse) caused no changes in either small intestinal or gastroduodenal permeability. The effect of zonulin was completely reversible, since both small intestinal (FIG. 4C) and gastroduodenal (FIG. 4D) permeability returned to pre-challenge values within 48 h. The differences in lacman or sucrose fractional excretion are shown as percentage of permeability change between the value of 2 days after the challenge and the challenge day. *Lacman P<0.0024 compared to both BSA control and 2-chain HP2; *Sucrose P<0.0049 compared to both BSA control and 2-chain HP2 (n=10 for each group of treatment).

FIGS. 5A-5D show the effect of zonulin on EGFR phosphorylation. In FIG. 5A zonulin at increasing concentrations was incubated on serum-starved Caco-2 cells. The cells were lysed, immunoprecipitated using anti-EGFR Ab, and processed for WB using anti-phospho EGFR (PY Plus) Ab. To ensure equal loading, the blots were stripped and re-probed for EGFR. Zonulin caused a dose-dependent increase in EGFR phosphorylation that reached a plateau at 3 ml/ml. In FIG. 5B zonulin at 10 ml/well was incubated either alone (lane 2) or in the presence of 5 μM of the EGFR-selective PTK inhibitor AG1478 (lane 3) on serum-starved Caco-2 cells. Cells exposed to media (lane 1) or AG1478 alone (lane 4) were used as additional controls. Zonulin caused an increase in EGFR phosphorylation that was completely abolished by the PTK inhibitor AG1478 (n=3 experiments). In FIG. 5C zonulin 10 mg/ml, either alone or in the presence of 5 μM of AG1478, was applied to the luminal side of C57BL/6 WT intestinal segments at a concentration of 10 μg/well and TEER measured at baseline (open bars) and 90 min post-incubation (closed bars). Zonulin caused a significant drop in TEER that was prevented by the presence of AG1478 (n=4 mice for each group).

FIG. 5D shows that the zonulin-induced EGFR phosphorylation was significantly reduced following treatment with two-chain mature HP2 (10 ml/ml) (lane 3) compared with single chain zonulin (lane 2). Lane 1 shows EGFR phosphorylation in cells treated with media alone.

FIGS. 6A-6B illustrate the effects of zonulin on EGFR phosphorylation and IP. FIG. 6A shows that zonulin-induced EGFR phosphorylation was decreased when PAR₂ was silenced. PAR₂ expression was silenced in Caco-2 using two different PAR₂ siRNAs. Cells were then treated with zonulin (10 mg/ml) or media control, lysed, immunoprecipitated using anti-EGFR Ab, and processed for WB with anti-phospho-EGFR PY-plus Ab. Zonulin-mediated EGFR phosphorylation was prevented by PAR₂ silencing. Equivalent protein loading and transfer was confirmed by stripping and reprobing the blots for EGFR. FIG. 6B shows that zonulin did not increase intestinal permeability in PAR₂^{-/-} mice. Segments of small intestine from both C57BL/6 WT and PAR2^{-/-} mice were mounted onto the microsnapwell system, exposed for 30 min to medium alone or to the medium containing 10 mg the purified recombinant zonulin, and transepithelial electrical resistance (TEER) monitored at time 0 (open bars) and after 90 min incubation (closed bars). The zonulin-induced drop in TEER observed in wild-type mice was ablated in PAR₂^{-/-} mice (n=5).

FIGS. 7A-7C illustrate serum zonulin levels and their correlation with intestinal permeability. In FIG. 7A CD patients showed higher serum zonulin levels compared to both their relatives and controls. In FIG. 7B similar results were obtained in TID patients. FIG. 7C shows that serum zonulin correlated with intestinal permeability evaluated by the LA/MA test.

FIGS. 8A-8B illustrate haptoglobin (HP) genotyping and phenotyping. FIG. 8A shows an agarose gel of 3 amplicons from 3 human subjects showing the 3 possible HP genotypes. FIG. 8B shows a Western immunoblotting using polyclonal anti-HP antibodies.

### DETAILED DESCRIPTION OF THE INVENTION

The following abbreviations may be used herein. Ab: Antibodies; EGFR: Epidermal Growth Factor Receptor; HP: Haptoglobin; IP: Intestinal Permeability; PAR: Proteinase Activating Receptor; TJ: Tight Junctions; WB: Western Blot; CD: celiac disease.

As used herein, the term “a” or “an”, when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, method steps, and/or methods of the invention. It is contemplated that any device, compound, composition, or method described herein can be implemented with respect to any other device, compound, composition, or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the term “contacting” refers to any suitable method of bringing one or more of the compounds described herein with or without one or more other therapeutic agents into contact with one or more cells. For in vivo applications, any known method of administration is suitable as described herein.

As used herein, the terms “effective amount”, “pharmacologically effective amount” or “therapeutically effective amount” are interchangeable and refer to an amount that results in an effect against cells in vitro or an improvement. Those of skill in the art understand that the effective amount may improve the patient's or subject's condition, but may not be a complete cure.

As used herein, the term “subject” refers to any target of the treatment.

The present invention is directed to a method for diagnosing an immune-mediated disease in a subject, comprising the steps of obtaining a biological sample from the subject and a healthy control; and in a single amplification step, performing a genotype amplification of a haptoglobin gene comprising the sample and the control, wherein an increase in copies of a haptoglobin 2 genotype compared to control correlates to a diagnosis and severity of the immune-mediated disease in the subject. The single step genotype amplification may be accomplished by using specific primers in exon 2 and exon 5 of haptoglobin 1 (HP1) that correspond to exons 2 and 7 of haptoglobin 2 (HP2), particularly, although not limited to, primer sequences of SEQ ID NO: 3 and SEQ ID NO: 4. After amplification, a determination of a monozygous genotype for haptoglobin 1 (HP1-1) is indicative of zero copies of zonulin gene and correlates to no disease. A determination of a heterozygous genotype for haptoglobin 2 (HP2-1) is indicative of one copy of zonulin gene and correlates to a diagnosis of the immune-mediated disease. A determination of a homozygous genotype for haptoglobin 2 (HP2-2) is indicative of two copies of zonulin gene and correlates to a more severe disease than diagnosed for HP2-1. Those biological samples as described supra are useful in using the genotyping method. Representative examples of immune-mediated diseases are as described supra.

The present invention also is directed to a single step method for determining severity of an immune-mediated disease in a subject, comprising identifying a genotype for haptoglobulin 2 (HP2) from an amplified haptoglobin gene in a biological sample from the subject, wherein a homozygous genotype for haptoglobin 2 is indicative of two copies of zonulin gene thereby correlating to a more severe disease than a heterozygous genotype that is indicative of one copy of zonulin. The specific primers, immune-mediated diseases and biological samples are as described supra.

The present invention is directed further to a method for diagnosing a disease associated with increased intestinal permeability in a subject, comprising the steps of: obtaining a biological sample from the subject; measuring an expression level of a pre-haptoglobin or glycoform thereof in the biological sample; and comparing the expression level of the pre-haptoglobin or glycoform thereof in the sample with an expression level of the same expressed in a control sample; wherein overexpression of the pre-haptoglobin or glycoform thereof compared to the control is indicative of the presence of the autoimmune disease. Representative examples of the biological samples useful in the diagnostic method are, but not limited to, blood serum, urine, stool, or a tissue biopsy. Particularly, the pre-haptoglobin may be pre-haptoglobin 2. The disease associated with increased intestinal permeability is an allergic, an inflammatory or an autoimmune disease. Representative example of the disease are as described supra.

In this method the pre-haptoglobin expression level is measured at the mRNA level. For example, in one aspect the pre-haptoglobin is pre-haptoglobin 2 and measuring the expression level thereof comprises isolating mRNA from the sample; and amplifying and quantifying pre-haptogobin 2 mRNA in the sample. Alternatively, the expression levels of the pre-haptoglobin and the glycoforms thereof is measured at the protein level. In another aspect of the present invention, measuring the expression level of the pre-haptoglobin or glycoform thereof comprises contacting the sample with an antibody directed against haptoglobin alpha or beta chain or a glycoform thereof; contacting the antibody-bound haptoglobin chain or antibody-bound glycoform thereof with another detection antibody specific to the pre-haptoglobin or the pre-haptoglobin glycoform thereof; and detecting and quantifying the pre-haptogobin protein or the pre-haptoglobin glycoform in the sample. In an alternate aspect measuring the expression levels of the pre-haptoglobin or the glycoform thereof comprises contacting the sample with a polyclonal or monoclonal antibody directed against the pre-haptoglobin or the glycoform thereof; and detecting and quantifying pre-haptoglobin protein or the glycoform thereof in the sample.

The present invention is directed further still to a method for diagnosing an autoimmune disease in a subject, comprising obtaining a biological sample from the subject; amplifying pre-haptoglobin 2 mRNA in the biological sample; and quantifying the pre-haptoglobin2 in the amplified product; where an increase in pre-haptoglobin-2 product compared to a control is indicative of the presence of the autoimmune disease. The method may utilize biological samples as described supra and is useful to diagnose an autoimmune disease as described supra, particularly celiac disease.

The present invention is directed further still to a method for diagnosing an autoimmune disease in a subject, comprising the steps of obtaining a biological sample from the subject; detecting pre-haptoglobin 2 protein in the biological sample; and quantifying the detected pre-haptoglobin 2 protein; where an increased level of pre-haptoglobin-2 in the sample compared to a control is indicative of the presence of the autoimmune disease. The method may utilize biological samples as described supra and is useful to diagnose an autoimmune disease, also as described supra, particularly celiac disease.

In one aspect, the pre-haptoglobin 2 is detected by contacting the biological sample with an antibody directed against haptoglobin alpha or beta chain; and contacting the antibody-bound haptoglobin with another detection antibody specific to prehaptoglobin 2. In an alternative aspect, detecting comprises contacting the biological sample with an antibody directed against prehaptoglobin 2.

Thus, after diagnosis, the immune-mediated, e.g., autoimmune, disease may be treated. The present invention therefore is directed to a method of treating an autoimmune disease, comprising the step of decreasing cell permeability leading to increased transepithelial electrical resistance. This method would be applicable to any autoimmune disease in which decreased cell permeability is desired. Representative cells in which cell permeability would be decreased include but are not limited to small intestinal cells or gastroduodenal cells. In one aspect, such a cell would have a decreased expression of zonulin mRNA. In a related aspect, this method further comprises the step of inhibiting epidermal growth factor receptor. A person having ordinary skill in this art would readily recognize known techniques to inhibit epidermal growth factor receptor to use in this method. For example, in a preferred embodiment, epidermal growth factor receptor is inhibited by administering an antibody directed against single chain zonulin. In another related aspect, this method further comprises the step of inhibiting PAR₂. A person having ordinary skill in this art would readily recognize known techniques to inhibit PAR₂.

In preferred embodiments of this method of the present invention, PAR₂ is inhibited using an antibody directed against single chain zonulin or using an siRNA. In another related aspect, this method further comprises the step of avoiding zonulin release by gliadin through CXCR3 receptor binding. Representative autoimmune diseases which may be treating using this method of the present invention include but are not limited to type I diabetes mellitus (T1D), systemic lupus erythematosus, celiac disease, ankylosing spondylitis, multiple sclerosis, rheumatoid arthritis, Crohn's disease, chronic kidney disease, or schizophrenia.

The present invention is further directed to a method of treating an autoimmune disease in an individual in need of such treatment, comprising the steps of inhibiting epidermal growth factor receptor; and inhibiting PAR₂. Using this method, cell permeability is decreased leading to increased transepithelial electrical resistance. Cell permeability may be decreased in any cell including but not limited to small intestinal cells or gastroduodenal cells. Typically, such cell will exhibit decreased expression of zonulin mRNA. Epidermal growth factor receptor and PAR₂ may be inhibited as described above. In an additional aspect, this method further comprises the step of inhibiting gliadin using any technique known to those of ordinary skill in this art, including anti-gliadin antibodies. Representative diseases which may be treated using this method of the present invention include but are not limited to autoimmune disease such as type I diabetes mellitus, systemic lupus erythematosus, celiac disease, ankylosing spondylitis, multiple sclerosis, rheumatoid arthritis, Crohn's disease, chronic kidney disease, or schizophrenia.

The present invention is further directed to a method for treating celiac disease in an individual in need of such treatment, comprising the steps of: administering an antibody directed against single chain zonulin thereby inhibiting epidermal growth factor receptor and inhibiting PAR₂. Using this method of the present invention, cell permeability is decreased leading to increased transepithelial electrical resistance. Representative cells include small intestinal cells or gastroduodenal cells but this method could be useful in many cell types. In a related aspect of this method, PAR₂ is further inhibited using an siRNA. In addition, this method may further comprise the step of inhibiting gliadin.

Increased intestinal permeability (IP) has emerged as a common, underlying mechanism in the pathogenesis of allergic, inflammatory, and autoimmune diseases. The characterization of zonulin, the only physiological mediator known to reversibly regulate intestinal permeability has remained elusive. Through proteomic analysis of human sera, the present invention identified human zonulin as the precursor for haptoglobin-2 (pre-HP2). While mature HP is known to scavenge free hemoglobin to inhibit its oxidative activity, no function has ever been ascribed to its uncleaved precursor form.

The present invention demonstrates that the single chain zonulin contains an EGF-like motif that leads to transactivation of EGF receptor (EGFR) via Proteinase Activated Receptor (PAR)₂ activation. Activation of these two receptors was coupled to increased intestinal permeability. siRNA-induced silencing of PAR₂ or the use of PAR₂^{-/-} mice prevented loss of barrier integrity. Proteolytic cleavage of zonulin into its α2 and β subunits neutralized its ability to both activate EGFR and increase intestinal permeability. Quantitative gene expression revealed that zonulin is overexpressed in the intestinal mucosa of subjects with celiac disease. This is the first example of a molecule that in its precursor form exerts a biological activity that is distinct from the function of its mature form.

These results, therefore, characterize zonulin as a novel ligand that engages a key signalosome involved in the pathogenesis of human immune-mediated diseases that can be targeted for therapeutic interventions. Thus the present invention provides methods of diagnosing and treating immune-mediated diseases, such as, but not limited to, allergic, an inflammatory or an autoimmune disease. Particular autoimmune diseases are type 1 diabetes, systemic lupus erythematosus, celiac disease, ankylosing spondylitis, multiple sclerosis, rheumatoid arthritis, Crohn's disease, chronic kidney disease, or schizophrenia. Preferably, diagnosis comprises determining a genotype of a haptoglobin gene in a subject having, suspected of having or at risk for an immune-mediated disease. Alternatively, diagnosis may comprise detecting and measuring or quantifying expression levels, for example, mRNA or protein or gene product levels of pre-haptoglobin or a glycoform thereof.

Upon diagnosis a therapeutic strategy that decreases cell permeability and concomitantly increases transepithelial electrical resistance may be planned and instituted. This may comprise one or more therapeutic steps designed to inhibit epidermal growth factor receptor and/or proteinase-activated receptor 2 while further inhibiting gliadin. For example, treatment may be effective by utilizing an antibody directed against zonulin, e.g., single chain zonulin, or by utilizing a small interfering RNA or small molecule inhibitor.

The following example(s) are given for the purpose of illustrating various embodiments of the invention and are not meant to limit the present invention in any fashion.

### EXAMPLE 1

### Human Serum Samples

Human sera from both healthy and CD volunteers were obtained from the Center for Celiac Research serum bank. All samples were depleted of albumin and IgG using commercially available kits (Enchant™ Life Science kit; Pall Corporation, Ann Arbor, Mich., USA) and IgG ImmunoPure immobilized protein G plus (PIERCE, Rockford, Ill., USA), respectively). The albumin- and IgG-depleted sera were analyzed by SDS-PAGE, 2-D electrophoresis, and WB analysis.

### EXAMPLE 2

### Human Haptoglobins

HP1-1 and HP2-2 extracted from human plasma were purchased from Sigma (St. Louis, Mo., USA). HP SDS-PAGE, both mono- and two-dimensional gel electrophoresis WB, and mass-spectrometry analyses were performed. HP deglycosylation was performed by addition of N-glycosidase F (PNGase F) according to the manufacturers instructions (Sigma, St Louis, Mo., USA).

### EXAMPLE 3

### SDS/PAGE and WB Analysis

Albumin- and IgG-depleted sera (50 micrograms per well), human HP1-1 (1 mg per well), and human HP2-2 (1 microgram per well) were resolved by SDS/PAGE under both denaturing and nondenaturing conditions on 18% or 12% SDS/PAGE Tris-Glycine gels (Invitrogen), respectively. The denaturing condition required addition of 30 microliters of Laemmli buffer to the samples, followed by a 5-min boiling step before SDS/PAGE. Proteins were either stained with SimplyBlue SafeStain solution (Invitrogen) or transferred onto a PVDF membrane (Millipore) and probed with either 5 micrograms/mL affinity purified rabbit polyclonal anti-Zot IgG Ab, which were previously shown to cross-react with purified human zonulin (1) using the ImmunoPure IgG (Protein A) Purification Kit (PIERCE), or with 2 micrograms/mL mouse monoclonal anti-human HP (Sigma) or 1 mg/mL rabbit polyclonal anti-human HP (Sigma) as the primary Ab. HRP-labeled polyclonal anti-rabbit IgG (1:5,000; Amersham) or anti-mouse IgG (1:10,000; Sigma) was used as a secondary Ab. Bands were detected with ECL Plus reagents (Amersham).

### EXAMPLE 4

### 2-DE Analysis and 2-DE WB

2-DE was performed using the ZOOM IPGRunner System (Invitrogen). Briefly, albumin and IgG depleted sera were added to the commercial sample rehydration buffer containing urea, detergent, reducing agent, ampholyte solution, and a dye (ReadyPrep Rehydration/Sample buffer; Bio-Rad) in a ratio of 1:2 to rehydrate the ZOOM STRIP pH 5.3-6.3 (Invitrogen) for 1 h at room temperature (RT). The strips were then loaded in the ZOOM IPGRunner Cassette (Invitrogen) to perform the isoelectric focusing (IEF). To fractionate samples, an isoelectric focusing step voltage protocol of 200 V for 20 minutes, 450 V for 15 minutes, 750 V for 15 minutes, and 2,000 V for 105 minutes was used. After the isoelectric focusing step, before the 2-DE SDS/PAGE, strips were equilibrated for 15 minutes in NuPAGE LDS Sample buffer (Invitrogen) containing NuPAGE Sample Reducing Agent and alkylated for 15 min in NuPAGE LDS Sample buffer containing freshly added iodoacetamide (125 mM; BioRad). 2-DE SDS/PAGE was run using NuNovex 4-20% Tris-GlycineZOOMGels (1.0 mm) in an immobilized pH gradient well (Invitrogen). Protein bands were visualized by SimplyBlue SafeStain solution (Invitrogen). Protein bands were transferred onto PVDF membrane (Millipore) and probed using affinity-purified [Immuno-Pure IgG (Protein A) Purification Kit; PIERCE] rabbit polyclonal zonulin cross-reacting anti-Zot IgG (5 micrograms/mL) as the primary antibody and anti-rabbit IgG (ECL Rabbit IgG, HRP-Linked; Amersham Biosciences) as the secondary antibody. Films were developed after exposure of the PVDF membrane with ECL detection reagent (Amersham Biosciences).

### EXAMPLE 5

### MS Analysis

In-gel tryptic digest for protein band identification was performed on gel bands prestained with SimplyBlue excised from the SDS/PAGE or 2-DE and analyzed by MS/MS to identify the protein using the protein sequencing/mass mapping facility at the Stanford Protein and Nucleic Acid Biotechnology Facility (Beckman Center, Stanford, Calif.).

### EXAMPLE 6

### Expression of the Zonulin/Pre-HP2 in Insect Cells

Human full-length cDNA clone encoding for the HP2 was purchased from OriGene (TC116954; accession no. NM_{—}005143; OriGene Technologies, Inc.). Recombinant baculoviruses containing WT human zonulin cDNA, with a 6×His tag at the C-terminus, were constructed using pDEST8 and the Bac-to-Bac baculovirus expression system (Invitrogen) according to the manufacturer's protocol. Zonulin was then transferred from the pENTR/D-TOPO vector into the pDEST8 through recombination using Gateway technology (Invitrogen). MAX Efficiency DH10Bac cells carrying bacmid DNA were transformed with pDEST8-zonulin. Recombinant bacmid was isolated from DH10Bac cells and transfected into Spodoptera frugiperda (Sf9) cells using Cellfectin reagent (Invitrogen) to generate recombinant baculoviruses. Sf9 cells were used for expression of zonulin protein. For protein expression of zonulin, Sf9 cells (3×10⁷) were grown in suspension flasks in SFM-900 III medium (Invitrogen) at 27° C. Cells were infected by recombinant baculoviruses at a multiplicity of infection of 3. At 72 hours after infection, Sf9 cells were collected by centrifugation for 10 min at 2,000×g. For purification of the zonulin, phosphate buffer (pH 7.5) and NaCl were added to the conditioned medium to final concentrations of 20 mM and 0.5 M, respectively (2). The solution was applied to a chelating sepharose (His-bind resin; Novagen) column charged with Ni2⁺ and then eluted with 200 mM imidazole and dialyzed into PBS. The purified human zonulin was aliquoted and stored at −80° C. until use.

### EXAMPLE 7

### Ex Vivo IP Studies by the Microsnapwell System

The effect of zonulin/pre-HP2 on ex vivo intestinal permeability was monitored in the microsnapwell system as described (3). Briefly, segments of small intestine from C57BL/6 WT mice were mounted onto the microsnapwell system, and their luminal side was exposed for 30 min to medium alone or to the medium containing increasing concentrations of the purified recombinant zonulin. TEER was measured at time 0 and at 30-min time intervals for a period of 2 h using a planar electrode (Endohm SNAP electrode attached to an Evom-G WPI analyzer; World Precision Instruments) and expressed in Ω/cm² after normalization. All the TEER microsnapwell experiments were performed on mouse small intestine with a baseline TEER value of 77.9±3.5Ω/cm² (n=23). In selected experiments, the effect of zonulin on TEER was monitored both under basal conditions and after pretreatment with the EGFR tyrosine kinase inhibitor AG1478. In another set of experiments, zonulin was tested both in C57BL/6 WT and PAR2−/− mice.

### EXAMPLE 8

### In Vivo IP

129/SvEv WT mice were randomized into 3 groups of 30 mice. They were acclimatized to the experimental techniques for 3 wk, by fasting the animals for 3 h, gavaging the animals with a sugar probe, and placing them in metabolic cages twice each wk. On the day of protein challenge, the animals received either 170 micrograms of the purified single-chain zonulin in a 60 microliter solution or a similar amount of purified 2-chain cleaved HP2, together with the sugar gavage as described (4). Mice were placed in metabolic cages and offered drinking water ad libitum for the following 22 h; during this time, their urine was collected, and the mice were then returned to conventional cages. Two days after the drug challenge day, mice were again placed in metabolic cages to measure their recovery from the treatment.

### EXAMPLE 9

### Knockdown of PAR2 Through RNA Interference

PAR2 expression in Caco-2 cells was silenced using 2 different PAR2 siRNAs [HSS103471 and HSS103473 (50 nM each); Invitrogen]. The cells were transfected following the manufacturer's instructions with the PAR2 siRNAs using DharmaFECT1 transfection reagent (Dharmacon) in a 10-cm plate in the presence of 5% FCS for 24 h. PAR2 knockdown efficiency was confirmed by both WB and real-time PCR analysis.

### EXAMPLE 10

### Total RNA Extraction from Intestinal Biopsies

Total RNA was extracted using the TRizol RNA purification protocol. Briefly, each intestinal tissue specimen was homogenized in 1 mL of TRizol Reagent (Invitrogen) using the Polytron power homogenizer PT 3100 (KINEMATICA AG). RNA was extracted by adding 0.2 mL of chloroform. After shaking the tube vigorously by hand for 15 sec, samples were incubated at RT for 5 min and centrifuged at 15,000×g for 15 min at 4° C. (Marathon 21000R centrifuge; Fisher Scientific). After transferring the RNA-rich aqueous phase to another tube, RNA was precipitated by adding 0.5 mL of isopropyl alcohol per 1 mL of TRizol Reagent used for the initial homogenization. Samples were incubated at RT for 10 min and centrifuged at 15,000×g for 10 min at 4° C. After removing the supernatant, the RNA pellet was washed once with 75% ice-cold ethanol, adding at least 1 microliter of 75% ethanol per 1mL of TRizol Reagent used for the initial homogenization. The pellet was air-dried for no more than 2 min, dissolved in 20 microliters of RNase-free water, and stored at −80° C. The RNA concentration was read at 260 nm by spectrophotometer (DU530, UV/vis; Beckman Coulter). The 260:280 ratio was determined for each sample.

### EXAMPLE 11

### cDNA Synthesis

Two micrograms of total RNA was reverse transcribed with the High-Capacity cDNA Archive Kit according to manufacturer's instructions (Applied Biosystems).

### EXAMPLE 12

### PCR Amplification of HP in Human Intestinal Biopsies

Aliquots of the cDNA were utilized for PCR of fragments specific to HP2 using the following primer pairs, which were specifically designed to cover different exons: forward primer (exon 5) 5′-ATGGCTATGTGGAGCACTCG-3′ (SEQ ID NO: 1) and reverse primer (exon 7) 5′-TACAGGGCTCTTCGGTGTCT-3′ (SEQ ID NO: 2). PCR was performed with 0.1 micrograms of cDNA, 2.5 units of TaqDNA polymerase (Promega), 0.2 mM dNTP mix, 0.5 mM each primer, 5 mM MgCl2, and 1:10 volume of 10 microliters PCR standard buffer (Promega). The PCR was run in the thermal cycler (Thermo Electro Corporation). After an initial 1 min of denaturation at 94° C., 30 cycles comprising 30 sec at 94° C. (denaturation), 30 sec at 58° C. (annealing), and 30 sec at 72° C. (extension) were completed, followed by a 10-min final extension at 72° C. The PCR products were then separated on a 2% agarose gel, stained with ethidium bromide, excised out of the gel, purified using a gel band purification kit (Amersham Biosciences), and sequenced by a 3730xl DNA Analyzer (Applied Biosystems).

### EXAMPLE 13

### Real-Time PCR with the TaqMan Procedure

Real-time PCR was performed on the cDNA from only HP2-2 or HP2-1 phenotype subjects and was performed with HP2-specific gene primers and probes (product ID: Hs00978377_m1) and housekeeping 18S (product ID: Hs99999901_S1 (Applied

Biosystems). The reaction was performed with TaqMan Universal PCR Master Mix (Applied Biosystems, manufactured by Roche) and run on the 7500 Fast Real-Time PCR System (Applied Biosystems). All reactions were performed in duplicate. Relative gene expression was calculated using the comparative Ct method with 18S as a housekeeping gene. The fold change in zonulin mRNA expression in active CD patients and CD patients on a GFD diet relative to zonulin mRNA expression in non-CD controls after normalization to 18S mRNA was recorded.

### EXAMPLE 14

### Human Zonulin-PreHP2 Cloning and Expression in a Baculovirus Expression System and its Cleavage by Proteases

Recombinant zonulin/preHP2 protein production using a baculovirus system and its purification are described above. Purified single chain zonulin was subjected to proteolytic cleavage using the serine proteases indicated, resolved by SDS-PAGE, and then stained with SimplyBlue™ SafeStain solution (Invitrogen, Carlsbad, Calif., USA). For generation of two chain HP2, single chain zonulin was exposed to trypsin-agarose beads (Sigma T-1763) for 20 minutes at 25° C. The beads were removed by centrifugation, and the effectiveness of the removal of trypsin confirmed by assay of trypsin peptidase activity against the substrate Glu-Gly-Arg-pNA (Bachem Bioscience).

### EXAMPLE 15

### Ex Vivo and In Vivo IP Studies

The effect of zonulin on ex vivo and in vivo intestinal permeability was performed as described (8, 14) and reported above. To determine whether zonulin can activate EGFR, increasing concentrations of either zonulin or two-chain mature HP2 were added for increasing exposure times to serum-starved, high EGFR-expressing Caco-2 cells. The cells were lysed and processed for WB with anti-phospho EGFR (Y1068) Ab (Cell Signaling Technol. Inc.) as reported (40). Experiments were repeated in the presence of 5 μM of the EGFR-selective PTK inhibitor AG1478 (Calbiochem, Gibbstown, N.J., USA).

### EXAMPLE 16

### Zonulin Gene Sequencing and Quantification from Intestinal Tissue from Celiac Disease (CD) and Non-CD Patients

Samples of small-intestine mucosae were obtained from the second/third portion of the duodenum from subjects undergoing a diagnostic upper gastrointestinal (GI) endoscopy. Subjects included were 10 healthy controls, 7 patients with active CD at diagnosis, 3 patients with CD on treatment with a gluten-free diet for at least 6 months. All patients had clinical indications for the procedure and gave their informed consent to undergo an additional biopsy for the purpose of this study. The study protocol was approved by the Ethics Committee of the University of Maryland. The small-intestine biopsies were immediately collected in RNAlater RNA Stabilization Reagent (Qiagen, Valencia, Calif., USA) and stored at −20° C. until processed. Total RNA extraction, cDNA synthesis, and real time PCR are described above.

All values are expressed as mean±SE (standard error). The analysis of differences was performed by two-tailed Student's t tests to test differences between two groups for either paired or unpaired varieties. Multi-variate analysis was performed where appropriate. Values of P≦/0.05 were regarded as significant.

### EXAMPLE 17

### Characterization of Zonulin from CD Human Sera and Human HP Preparations

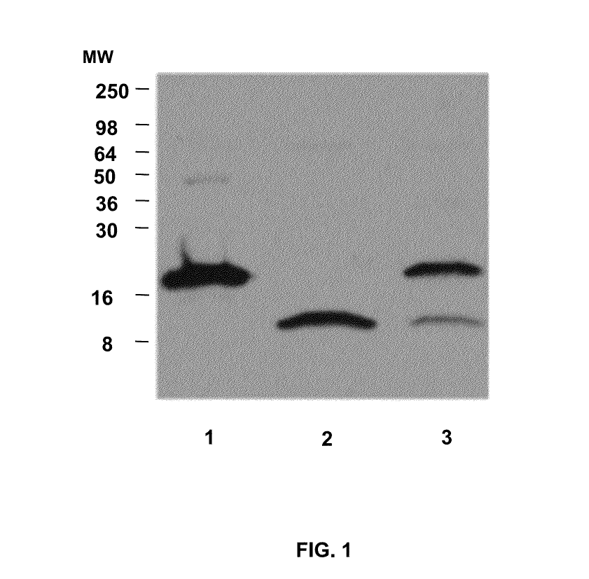

Since zonulin is detected in human sera by a zonulin cross-reacting anti-Zot Ab (Ab)-based ELISA (7-10) and is increased in patients with CD compared to normal controls (10), Western analysis was initially used to detect zonulin immunoreactivity of proteins in albumin- and IgG-depleted sera from CD subjects. These sera displayed two major protein bands with apparent molecular weights of 18 and 9 kDa (FIG. 1). Three distinct patterns of reactivity were identified in CD sera: a 18 kDa protein band (FIG. 1, lane 1), a 9 kDa protein band (FIG. 1, lane 2), and both 9 and 18 kDa protein bands (FIG. 1, lane 3). Of note, ˜45 kDa band was detected only in sera that displayed the single 18 kDa band (FIG. 1, lane 1), but not detected in sera with either the 9 kDa band or both bands (FIG. 1, lane 2 and 3). Two-dimensional gel electrophoresis (2-DE) of sera from CD patients who expressed the 18 kDa band revealed two zonulin immunoreactive spots that were subjected to MS/MS mass spectrometry analysis. The 18 kDa spot was identified as the a2 chain of HP2 (accession no. Gl:223976) and the 9 kDa spot as the al chain of HP1 (accession no. Gl:3337390). A random screening of 14 sera from CD patients revealed that 7% were HP1 homozygous, 57% HP1/HP2 heterozygous, and 36% HP2 homozygous.

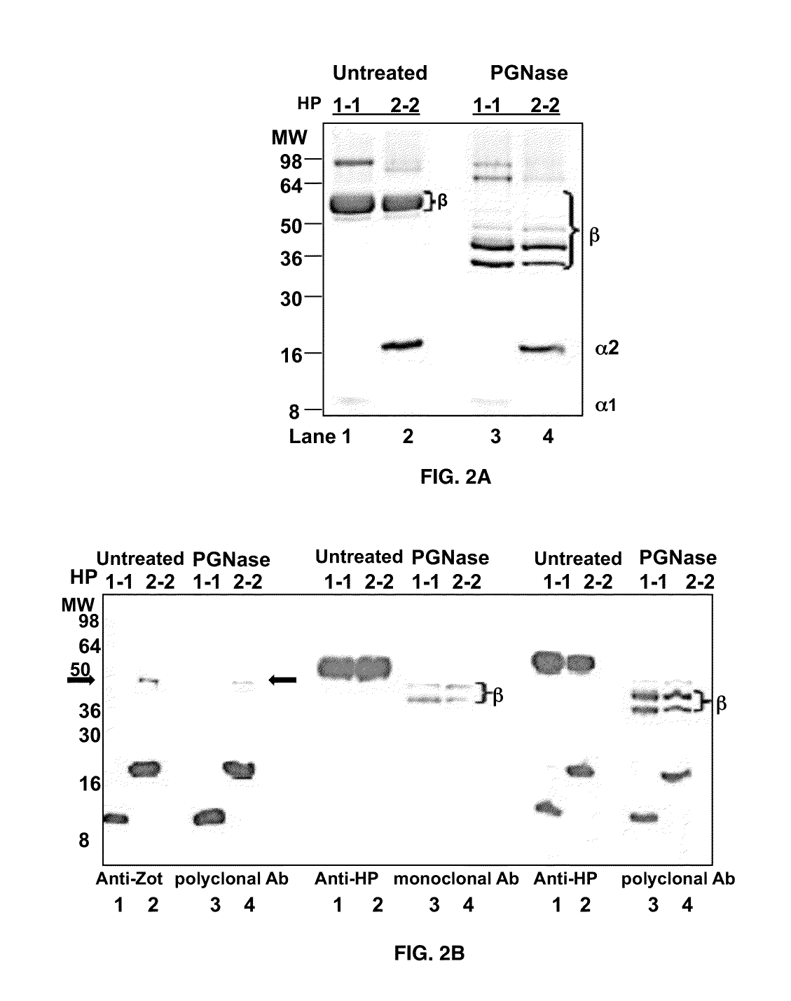

To confirm the identity of the immunoreactive bands recognized by the polyclonal zonulin-cross reacting anti-Zot IgG Ab in human CD sera, commercially purified preparations of human HP from subjects homozygote for either HP1 (HP1-1) or HP2 (HP2-2) were simultaneously resolved on a single gel by SDS-PAGE and analyzed by Coomassie staining (FIG. 2A). As expected, the al-chain of HP1-1 exhibited a MW of ˜9 kDa (FIG. 2A, lane 1), while the a2-chain of HP2-2 was ˜18 kDa (FIG. 2A, lane 2). Due to its glycosylation, the b chain exhibited a MW of ˜52 kDa in both HP1-1 and HP2-2 preparations (FIG. 2A, lanes 1 and 2). After a 3 hour deglycosylation reaction with N-glycosidase F (PGNase F), the b chain of both HP1-1 and HP2-2 ran as multiple bands below 52 kDa presumably due to varying degrees of deglycosylation (FIG. 2A, lanes 3 and 4). As anticipated, after glycosidase treatment, no changes in gel mobility for either the α1-chain of HP1-1 (FIG. 2A, compare lanes 1 and 3) or the α2 chain of HP2-2 (FIG. 2A, compare lanes 2 and 4) were evident.

FIG. 2B presents immunoblots of commercially available purified homozygous HP1-1 and HP2-2 proteins both before and after deglycosylation. Proteins were run simultaneously on a single gel and immunoblotted with polyclonal zonulin-cross reacting anti-Zot Ab (FIG. 2B, left), monoclonal anti-glycosylated β chain HP (FIG. 2B, center), or polyclonal anti-HP Ab (FIG. 2B, right). Anti-Zot Ab reacted strongly with both the HP1-1 α1 chain and the HP2-2 α2 chain and revealed an additional band at ˜45 kDa present in the HP2-2, but not in the HP1-1 preparations (FIG. 2B, left, lanes 2 and 1, respectively).

As expected, the monoclonal anti-HP antibody, raised against the ˜52 kDa HP β glycosylated subunit, recognized only the β chain of either HP1-1 or HP2-2 (FIG. 2B, center, lanes 1 and 2, respectively), while the polyclonal anti-HP Ab recognized epitopes of the α1, α2 and β chains of both HP1-1 and HP2-2 (FIG. 2B, right panel, lanes 1 and 2, respectively). FIG. 2B also shows immunoblotted HP1-1 and HP2-2 preparations after deglycosylation using the same three Ab. The pattern of reactivity of the three Ab tested for the non-glycosylated 9 kDa α1 and the 18 kDa α2 subunits did not change after deglycosylation (FIG. 2B, all, lanes 3 and 4, respectively). However, deglycosylation caused the expected gel mobility shift of the β chain in both HP1-1 and HP2-2. The monoclonal anti-HP Ab (FIG. 2B, center, lanes 3 and 4) recognized only 2 incomplete deglycosylated β chain bands, while the polyclonal anti-HP Ab recognized also the completely deglycosylated ˜36 kDa β chain (FIG. 2B, right, lanes 3 and 4). The 45 kDa band that was present only in the HP2-2 preparation and recognized by anti-Zot Ab did not show any change in gel mobility upon deglycosylation, but it appeared less intense (FIG. 2B, left, lane 4). MS/MS analysis and NH₂-terminal sequencing of this 45 kDa protein band performed on two distinct samples analyzed at different times identified this protein as the human HP2 precursor (pre-HP2, Accession No. P00738). The combined MS/MS analysis covered a total of 49.8% of non-overlapping protein and 13 unique peptides spanning the entire protein sequence. Therefore, in addition to α1 and α2 chains, the anti-Zot Ab recognize the uncleaved single chain pre-HP2, but not the β chain.

These results suggest that the anti-Zot Ab used to measure serum zonulin by ELISA should supposedly detect the highly abundant HP1 and HP2 proteins, as well as pre-HP2. However, the amount of serum zonulin detected by ELISA is in the ng/ml range (11), while the entire HP pool in serum is in the mg/ml range (12). To address this apparent discrepancy, the WB analysis of both human sera and purified HPs was repeated under non-denaturing conditions using anti-Zot Ab. The WB showed a series of bands in HP2-2 phenotype sera and in commercially purified HP2-2, while no bands were detected in either HP1-1 phenotype sera or in commercial purified HP1-1. Conversely, the anti-HP polyclonal Ab, that did not recognize the uncleaved pre-HP2, detected bands both in commercially purified HP1-1 and HP2-2 preparations. Combined, these data suggest that under non-denaturing conditions, the anti-Zot Ab detect only the single chain pre-HP2, but not the two-chain mature HPs, further supporting the notion that the single chain pre-HP2, but not its cleaved two-chain mature form, corresponds to the zonulin molecule.

### EXAMPLE 18

### Ex Vivo Effect of Recombinant Zonulin on TEER in Mouse Small Intestine Mounted in the Micro-Snapwell System

The primary translation product of the mammalian HP2 mRNA transcript is a polypeptide that dimerizes co-translationally and is proteolytically cleaved while still in the endoplasmic reticulum by the serine protease, Cr1LP (13). Conversely, zonulin is detectable in human serum as uncleaved pre-HP2 (see above). To confirm the identification of zonulin as the single chain pre-HP2 and not the cleaved mature two chain HP2, recombinant pre-HP2 was expressed by inserting the pre-HP2 cDNA into an insect cell vector and expressed it using a baculovirus expression system. Highly purified recombinant pre-HP2 was obtained that was recognized by the anti-Zot polyclonal Ab similarly to FIG. 2B and that migrated at an apparent MW of ˜53 kDa due to the 6×His tag attached at the C-terminus. The single chain pre-HP2 was then subjected to proteolytic cleavage using a series of serine proteases. Matriptase, urokinase, thrombin, and plasma kallikrein did not cleave pre-HP2, while plasmin caused complete degradation of the protein. In contrast, treatment with the intestinal serine protease trypsin led to the appearance of two major bands that migrated with molecular weights compatible with the α2 and β subunits of zonulin. NH₂-terminal sequencing of these 2 bands showed the 2 proteins to be identical the pre-H2 α2 and β chains cleaved at the predicted Arg¹⁶¹ cleavage site. The intact single chain pre-HP2 and the cleaved two-chain mature HP2, obtained after trypsin digestion, were both tested for their biological activities in the studies below.

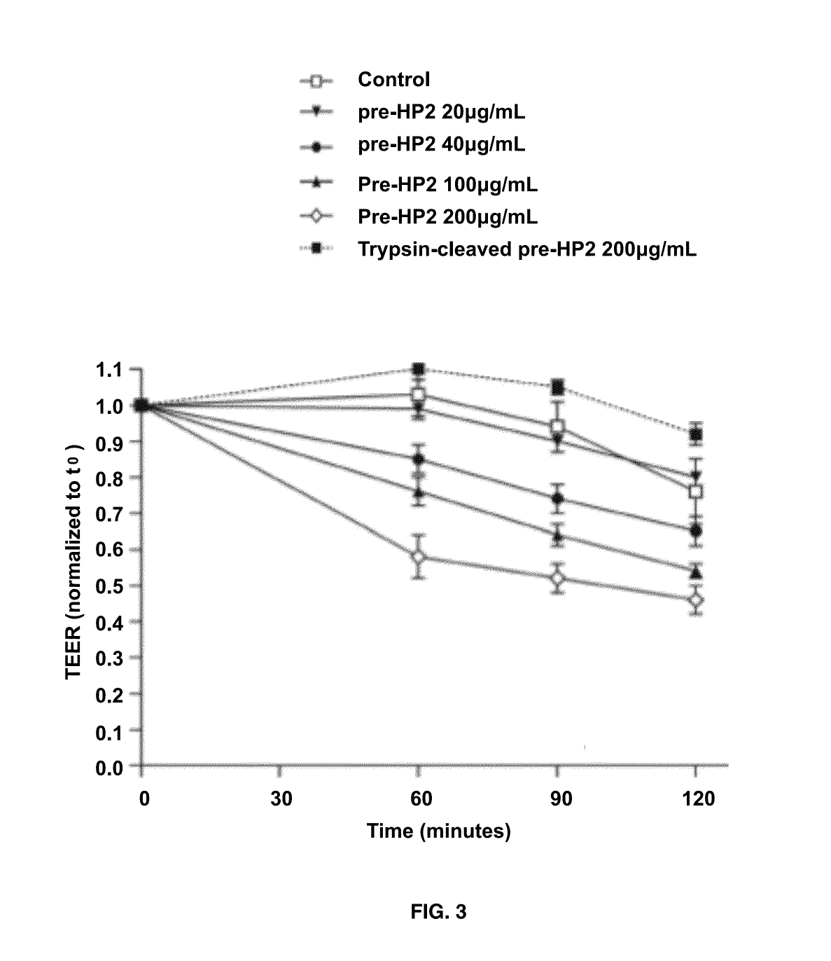

Recombinant pre-HP2 (from now on defined as zonulin) was applied to WT C57BL/6 murine small intestine segments mounted in microsnapwells. Recombinant single chain zonulin added to the mucosal (luminal) aspect of mouse intestinal segments decreased transepithelial electrical resistance (TEER), i.e., increased permeability, when applied at concentrations ≧40 μg/ml (FIG. 3). In contrast, no consistent TEER changes were detected when the trypsin-cleaved two chain HP2 was tested (FIG. 3).

### EXAMPLE 19

### In Vivo Effect of Recombinant Zonulin on Mouse Gastrointestinal Permeability

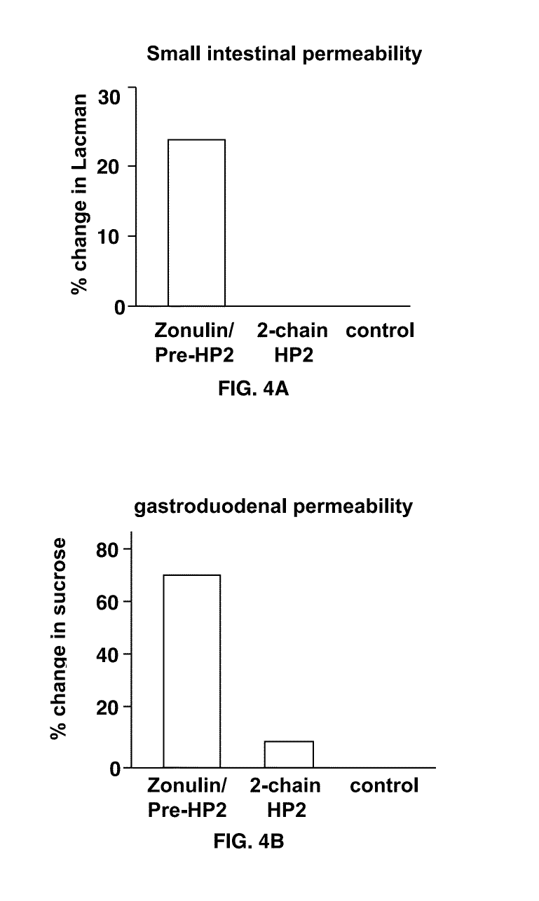

To establish whether zonulin might alter intestinal permeability in vivo, mice were gavaged with the single chain recombinant pre-HP2 protein (170 mg/mouse), and gastroduodenal and small intestinal permeability tested using specific sugar probes (sucrose and lactulose/mannitol, respectively) as described (14). Zonulin/preHP2 increased both small intestinal (FIG. 4A) and gastroduodenal (FIG. 4B) permeability compared to bovine serum albumin (BSA)-treated controls. Gastroduodenal and small intestinal permeability each returned to baseline within 48 hours following exposure to zonulin/preHP2 (FIGS. 4C-4D).

To determine whether the two-chain mature HP2 affected intestinal permeability, the in vivo experiments described above were repeated by administering two-chain proteolytically cleaved protein. In contrast to the single chain zonulin, two-chain HP2 (170 mg/mouse) failed to alter either gastroduodenal or small intestinal permeability compared to BSA-treated controls (FIGS. 4A-4B). Combined, these data indicate that the single chain zonulin, but not its two-chain mature HP2 form generated by proteolytic cleavage, retains the reversible permeating activity reported for zonulin.

### EXAMPLE 20

### Zonulin mRNA Expression and Quantification in Human Intestinal Mucosae

Using specific primers and the cDNA of human intestinal biopsies from zonulin positive subjects, a 686 by fragment was amplified, 144 bp of which belongs to the α-chain and 542 bp belonging to the β-chain of both HP1 and HP2 genes. Sequencing of this fragment confirmed its identity as HP, but HP1 could not be distinguished from HP2 because of the common sequence in the amplified region. To overcome this and to specifically quantify the expression of the zonulin gene in the human intestine, cDNA obtained from the intestinal mucosae of healthy individuals (n=10), celiac disease patients (disease in acute phase (n=7), and CD patients disease in remission following a gluten-free diet (GFD) (n=3) were analyzed by real-time PCR using primers and probes specific for the α2 chain. Compared to healthy individuals, zonulin mRNA expression was increased in the intestinal mucosae of celiac disease subjects with active disease (3-fold increase, P<0.05). Intestinal mucosae of three celiac subjects adhering to a gluten-free diet showed only 1.5 fold increase zonulin expression compared to controls.

### EXAMPLE 21

### Recombinant Zonulin Increases Tyrosine Phosphorylation of EGFR

Gliadin, a glycoprotein present in wheat and several other cereals and the environmental trigger responsible for the autoimmune damage of the small intestine typical of celiac disease (15), fully reproduces the effects of EGF on the actin cytoskeleton (16), effects that are very similar of zonulin (7, 10, 16). Furthermore, structural analysis revealed that the pre-HP-2 β chain includes an EGF motif that contains 6 spatially conserved cysteine residues that form 3 intramolecular disulfide bonds necessary for EGF-like activity.

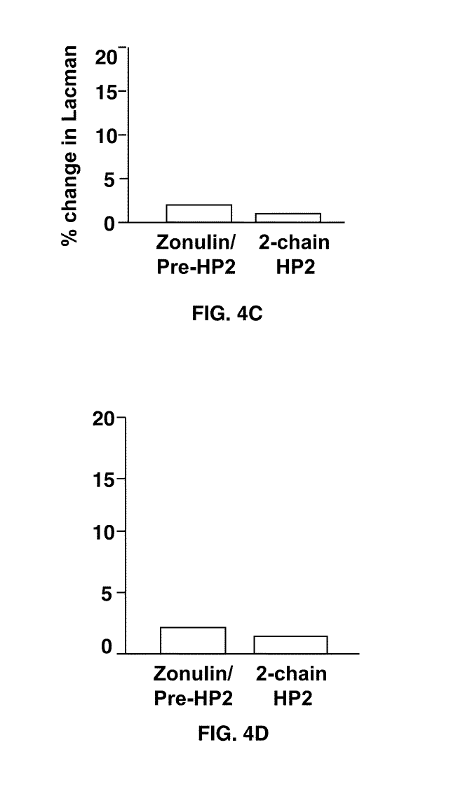

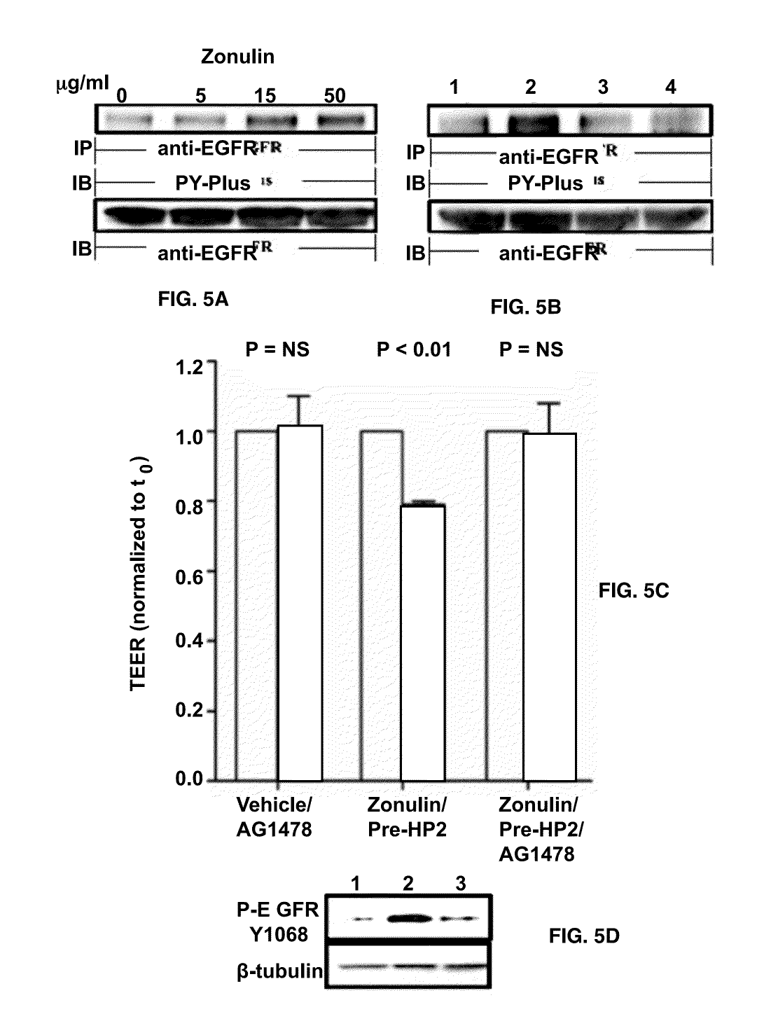

To determine whether zonulin can activate EGFR, increasing concentrations of baculovirus-derived, recombinant zonulin were added to Caco-2 intestinal epithelial cells. The cells were lysed, immunoprecipitated with anti-EGFR Ab, and processed for phosphotyrosine immunoblotting (PY-Plus). At concentrations ≧3 mg/ml, zonulin increased tyrosine phosphorylation of EGFR (FIG. 5A). To further establish the role of EGFR in zonulin-induced alterations in TEER, both in vitro and ex vivo experiments described above were performed in the presence of the EGFR-selective PTK inhibitor, AG1478. Pre-incubation of Caco-2 cells for 2 h with the EGFR selective protein tyrosine kinase inhibitor, AG1478 (5 mM), prevented zonulin/preHP2-induced EGFR phosphorylation on Y1068 (FIG. 5B). Similarly, pretreatment with AG1478 abolished TEER reduction in response of zonulin (FIG. 5C). Finally, trypsin digestion of zonulin dramatically reduced its ability to activate EGFR (FIG. 5D). Combined, these data suggest that the single chain zonulin activates EGFR and induces an EGFR-driven decrease in TEER, whereas the cleaved two-chain HP2 fails to both activate EGFR and to increase IP.

### EXAMPLE 22

### Recombinant Zonulin-Induced EGFR Activation and TEER Changes Are PAR₂-Dependent

Zot active peptide FCIGRL (AT1002) has structural similarities with the PAR₂-Activating Peptide (AP), SLIGRL, and causes PAR₂-dependent changes in TEER (17), a finding that was demonstrated in WT, but not PAR2^{-/-} mice. Further, several G protein coupled receptors (GPCR), including PAR₂, transactivate EGFR (18). Since Zot and zonulin share a similar mechanism of action (6) and the zonulin protein sequence contains a Zot-like and PAR₂ AP-like motif in its β chain (FCAGMS), whether zonulin-induced EGFR activation might be PAR₂-dependent was determined.

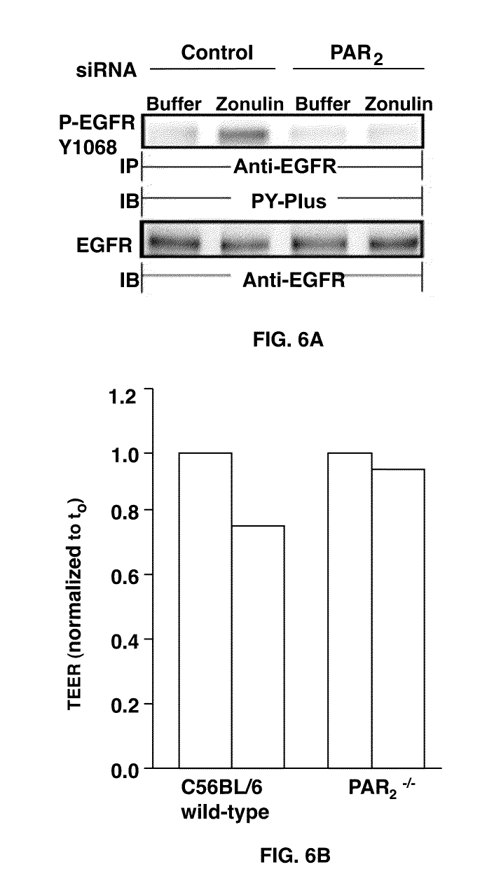

siRNA-induced silencing of PAR₂ in Caco-2 cells diminished EGFR Y1068 phosphorylation in response to recombinant zonulin (10 mg/ml) (FIG. 6A), compatible with PAR₂-dependent transactivation of EGFR.

To further establish a role for PAR₂ in EGFR activation in response to zonulin, small intestinal barrier function was studied in the microsnapwell system using segments isolated from either C57BL/6 WT or PAR_{2 -/-} mice. As anticipated, recombinant zonulin decreased TEER in intestinal segments from C57BL/6 WT mice, while it failed to reduce TEER in small intestinal segments from PAR₂^{-/-} mice (FIG. 6B), so linking zonulin-induced PAR₂-dependent transactivation of EGFR with barrier function modulation.

The present invention identified zonulin as the precursor of HP2. Mature human HPs are heterodimeric plasma glycoproteins composed of a and β polypeptide chains that are covalently associated by disulfide bonds and in which only the β chain is glycosylated (19). Unlike the β chain (36 kDa), the a chain exists in two forms, i.e., α1 (˜9 kDa) and α2 (˜18 kDa). The presence of one or both of the 2 chains results in the three phenotypes, HP1-1, HP2-1, and HP2-2. These HP variants evolved from a mannose-binding lectin-associated serine protease (MASP) (12, 20), with the a chain containing a complement control protein and the β chain a catalytically dead chymotrypsin-like serine protease domain (21-24). Other members of the MASP family include a series of plasminogen-related growth factors (EGF, HGF, etc.) involved in cell growth, proliferation, differentiation, migration, and disruption of intercellular junctions. Despite this multidomain structure, the only function assigned to HPs, to date, is to bind Hb to form stable HP-Hb complexes thereby preventing Hb-induced oxidative tissue damage (25). No function has ever been described for their precursor forms.

HPs are unusual secretory proteins in that their precursor proteins, instead of being cleaved in the trans-Golgi complex, are proteolytically processed by complement C1r-like protease (Cr1LP) in the endoplasmic reticulum (13). Of interest, the endoplasmic reticulum fraction was the cellular fraction in which the highest zonulin concentrations were detected (9).

Since the key biological effect of zonulin is to regulate intercellular TJ function (7, 9-11), recombinant pre-HP2 was examined in intestinal permeability assays. Pre-HP2 dose- and time-dependently reduced TEER across murine small intestinal mucosa both ex vivo and in vivo. The observation that zonulin lost its permeating activity after cleavage into its two a2 and β subunits further supports the notion that zonulin/pre-HP-2 and mature two-chain HP2 exert distinct biological functions. The importance of protein conformation in dictating HP protein function is further supported by the finding that zonulin-cross reactive anti-Zot Ab recognized the HP1 a1 chain under denaturing conditions (FIGS. 1A and 2B), but failed to recognize non-denatured HP1. Combined, these data confirm the identity of zonulin as pre-HP2.

The NH₂-terminal amino acid sequence of zonulin has striking similarities with the light chain of human g globulins (7), a similarity also noted for HP (26). Clearance of the HP-Hb complex can be mediated by the monocyte/macrophage scavenger receptor, CD163 (25). Clustal W dendogram analysis showed a region in the zonulin b chain just upstream of the CD163 binding site with the following gamma globulin-like consensus motif: QLVE-V-P. Discrepancies between the previously reported zonulin sequence and this pre-HP2 consensus motif may be due to intra-species differences.

Zonulin contains growth factor-like repeats. Like zonulin, growth factors affect intercellular tight junction integrity (27, 28). The present invention shows that the single chain zonulin, but not its cleaved mature form, transactivates EGFR via PAR₂ and that its effect on TEER is prevented by pharmacological inhibition of EGFR or siRNA-induced PAR₂ silencing. This suggests that the growth factor motif in the single chain zonulin, but not in the mature two-chain HP2, has the molecular conformation required to induce tight junctions disassembly by indirect transactivation via PAR₂.

Gliadin, the environmental trigger of CD, reportedly reproduces the effects of EGF on the actin cytoskeleton (16). These effects are very similar to the effects reported for zonulin (7). Gliadin binds to the CXCR3 chemokine receptor (29) and this interaction is coupled to zonulin-pre-HP2 release from both intestinal cells (9) and whole intestinal tissues (10). Hence, it is likely that the gliadin-related EGF effects are mediated through zonulin release. Intestinal bacterial colonization is also a stimulus for zonulin release (8). Gliadin and microorganisms both cause polarized, luminal secretion of zonulin (8). Therefore, studies were focused on early zonulin action, i.e., its activity at intestinal luminal side. This approach may appear counterintuitive, given the observation that both EGFR and PAR₂ are expressed basolaterally (3, 30). However, evidence exists that they also are apically expressed (31). The fact that zonulin exerted a permeating effect, both in ex vivo and in vivo, when applied to the luminal aspect of the intestinal mucosa does not dispute the possibility that the protein acts basolaterally as well. When environmental triggers (i.e. bacteria, gluten) are present in the intestinal lumen, zonulin is released from enterocytes, a process that is mediated, at least for gliadin, by CXCR3 (29). Following zonulin release and subsequent increase in intestinal permeability, these triggers can reach the submucosa where zonulin-expressing immune cells can present zonulin to the basolateral side. A similar bilateral action has been reported for mucosal mast cell protease II, another serine protease that controls intestinal permeability acting both from luminal and serosal sides (32).

The role of both EGFR and PAR₂ in regulating epithelial permeability has been previously reported (33, 34). However, the present invention provides the first evidence that the two receptors work cooperatively to regulate small intestinal permeability.

It has been reported that zonulin is upregulated during the acute phase of celiac disease (9, 10). Using HP-specific primers, the present invention reports for the first time the expression of zonulin mRNA in human intestine. Furthermore, real time PCR experiments showed that zonulin expression was increased in celiac disease patients compared to normal controls. The enhanced expression of zonulin correlated with disease activity as celiac disease patients who were on a gluten-free diet showed mean values for zonulin expression that were intermediate to active celiac disease patients and normal controls. Interestingly, Papp and co-workers recently reported that a polymorphism in the HP gene represents a novel genetic risk factor for celiac disease development and its clinical manifestations (35).

The human plasma levels of pre-HPs are between 100 and 300 mg/100ml, with HP2-2 ranging between 100-260 mg/100 ml. Almost 8% of HPs are secreted in their pro-form (36), suggesting that under physiological circumstances 80-208 mg/ml of pre-HP2 are present in human plasma. Therefore, the concentrations of zonulin used herein are within physiological range and are most likely indicative of the signaling pathways activated when zonulin is upregulated during pathological processes. Besides celiac disease, elevated levels of zonulin have been reported in other autoimmune diseases, including type I diabetes (11), systemic lupus erythematosus (37), and ankylosing spondylitis (38), further delineating the importance of the zonulin pathway in the pathogenesis of autoimmune diseases. These findings, together with the observation that zonulin is overexpressed during the acute phase of several immune-mediated diseases and its blockage prevents the onset of the autoimmune response, suggest that zonulin contributes to the pathogenesis of these conditions, opening new paradigms in the pathobiology and treatment options of immune-mediated diseases.

### EXAMPLE 23

### Zonulin Phenotype in Celiac Disease (CD) and Type 1 Diabetes (T1D) and its Correlation with Intestinal Permeability

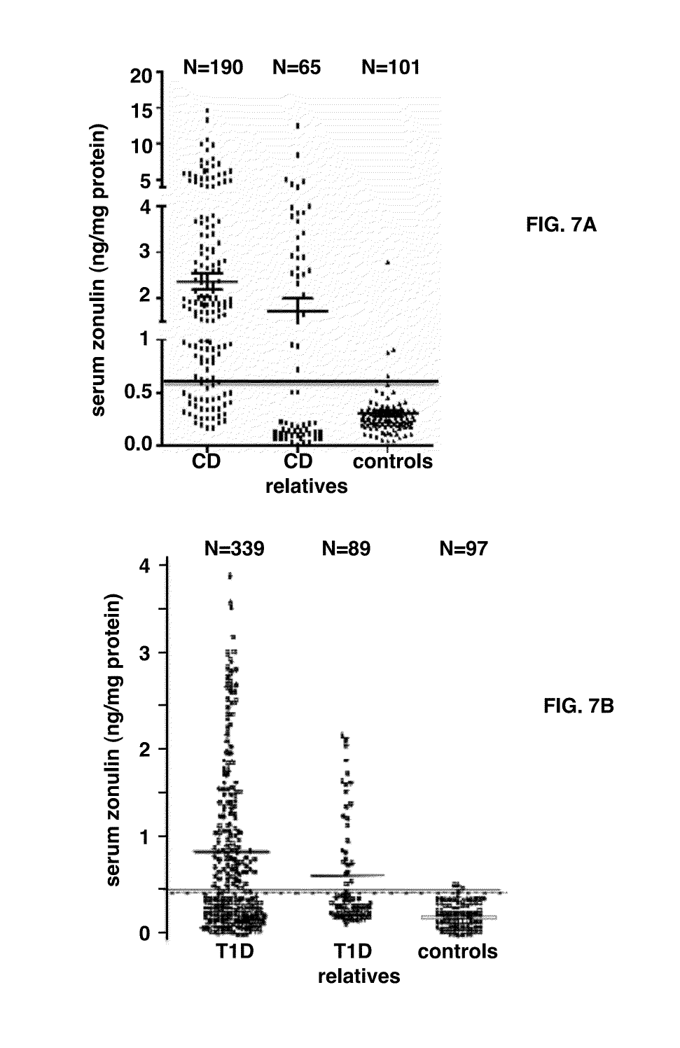

Using a serum zonulin ELISA developed with the support of this grant, we measured serum zonulin levels in both CD and T1D patients, their relatives, and age and sex-matched healthy controls. CD CD patients showed statistically higher serum zonulin levels (2.37±0.17 ng/mg protein) as compared to both their relatives (1.75±0.27 ng/mg protein, p=0.05) and control subjects (0.31±0.03 ng/mg protein, p<0.00001) (FIG. 7A). Eighty-one percent (154/190) of CD patients and 50% of their first-degree relatives (33/65) had serum zonulin levels that were 2 SD above the mean zonulin levels detected in age-matched healthy controls. Only 4.9% (5/101) of controls had zonulin levels 2 SD above the mean (p<0.01). Serum zonulin was higher in CD as compared to their relatives (p<0.00001). Similar results were obtained in T1D patients in which we detected increased serum zonulin levels (0.83±0.05 ng/mg protein) as compared to both their relatives (0.62±0.07 ng/mg protein, p=0.011) and control subjects (0.21±0.02 ng/mg protein, p<0.00001) (FIG. 7B). Forty-two percent (141/339) of T1D patients and 29% of their first-degree relatives (26/89) had serum zonulin levels that were 2 SD above the mean zonulin levels detected in age-matched healthy controls. Only 4% (4/97) of controls had zonulin levels 2 SD above the mean (p<0.01). Serum zonulin was higher in T1D as compared to their relatives (p=0.01). To establish whether serum zonulin levels correlated with intestinal permeability, lactulose (LA)/mannitol (MA) urine ratio was determined in both a subset of T1D subjects with documented zonulin up-regulation (N=36) and their relatives (N=56). Intestinal permeability correlated with serum zonulin (FIG. 7C).

### EXAMPLE 24

### New Method for HP Genotyping

In order to develop a high throughput method to establish the presence and copy number of HP2, i.e., zonulin, gene in a large number of samples, a new single step amplification method was developed using primers designed with Primer3 as previously described in exon 2 and exon 5 of HP1 corresponding to exons 2 and 7 of HP2. Briefly, genotyping was done with primers designed with Primer3 in exon 2 and exon 5 of HP1 corresponding to exons 2 and 7 of HP2 as follows: forward: TTTCTGGCTGCTAAGTTG (SEQ ID NO: 3) and reverse: AATGTCTTTCGCTGTTGC (SEQ ID NO: 4).

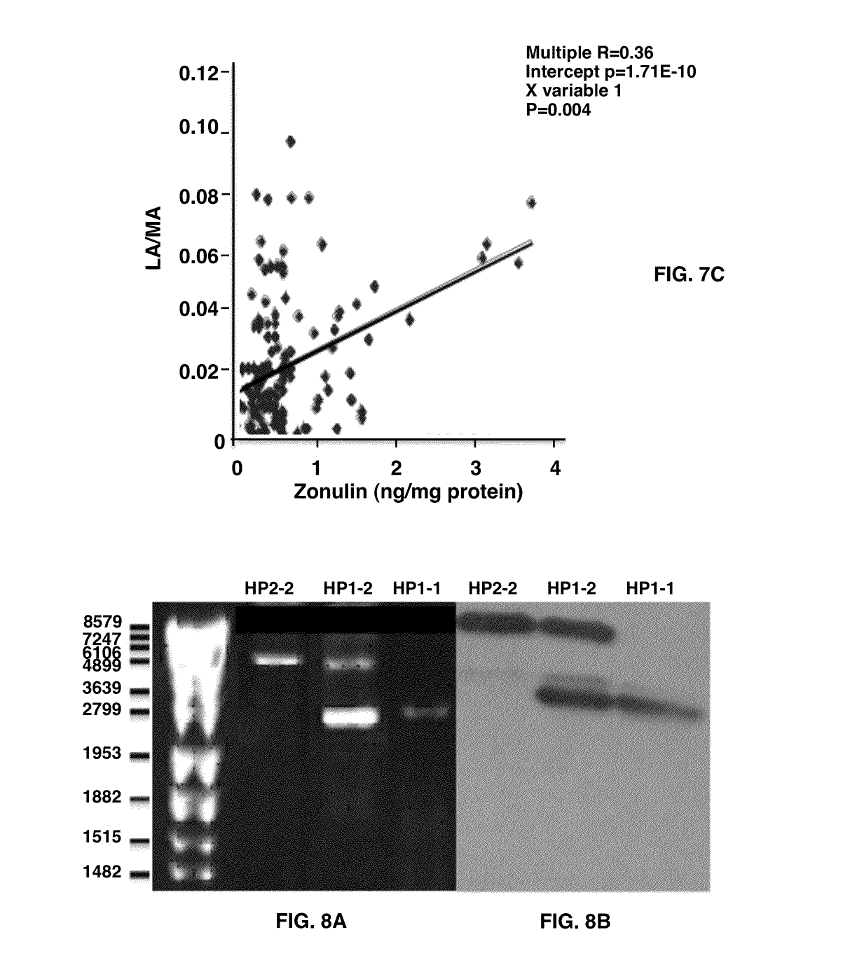

The PCR is set up in 50 μl reactions using the high fidelity PCR system from Roche Applied Science. The reactions contains 1 μl 10 mM nucleotide mix, 2.5 μl 300 nM of each primer, 5 μl of 50 ng/μl DNA, 5 μl buffer with 1.5 mM MgCL₂, 0.75 μl enzyme mix 2.6 U per reaction, and 33.25 μl nuclease free H₂O. The PCR is run with the following protocol: 1. 94° C.-2 m, 2, 30 cycles of 94° C.-15 s, 60° C.-90 s, 68° C.-2 m, 3. 72° C.-7 m. After PCR the amplicons are run on a 1% agarose gel and read under a UV bulb. The duplication in HP2 and the size difference allows differentiation of the two genotypes. The HP1 genotype will run at a size of 2.5 kb while the HP2 will run at 4.3 kb. Using this approach the 3 possible genotypes that matched with the corresponding phenotypes were identified (FIGS. 8A-8B).

### EXAMPLE 25

### HP2 (Zonulin) Allele Is Over-Represented in Immune-Mediated Diseases

To establish the distribution of HP1 and HP2 genes among CD patients and matched controls, a single step RT-PCR protocol was developed using specific primers in exon 2 and exon 5 of HP1 corresponding to exons 2 and 7 of HP2. After PCR the amplicons were run on a 1% agarose gel and read under a UV bulb. The HP1 genotype ran at the predicted size of 2.5 kb while the HP2 ran at 4.3 kb. The results showed that in CD patients HP1-1 genotype (0 copies of zonulin gene) was decreased, while the HP2-2 genotype (2 copies of zonulin gene) was increased as compared to healthy controls (Table 1). Interestingly, the percentage of HP 1-1 CD patients (0 copies of zonulin gene) was in the same range of the percentage of CD patients that tested negative by zonulin ELISA Similar distribution of the HP genes have been reported by other investigators in other immune-mediated diseases, including Crohn's disease, schizophrenia, and chronic kidney disease (CKD) (Table 1).

TABLE 1

| | Celiac | | Crohn's | | Schizo- | | | |
|---|---|---|---|---|---|---|---|---|
| | Disease | | Disease | | phrenia | | CKD | |
| Genotype | Cntr | Pts | Cntr | Pts | Cntr | Pts | Cntr | Pts |
| HP 1-1 | 20.6 | 7.1 | 23.9 | 10.1 | 20.6 | 7.1 | 23.9 | 10.1 |
| HP 1-2 | 43.5 | 35.7 | 44.0 | 46.2 | 43.5 | 35.7 | 44.0 | 46.2 |
| HP 2-2 | 35.9 | 57.2 | 32.1 | 43.7 | 35.9 | 57.2 | 32.1 | 43.7 |

The following references are cited herein.
1. Rook G A, Stanford J L (1998) Immunol Today 19:113-116.
2. Arrieta et al., (2006) Alterations in intestinal permeability. Gut 55:1512-1520.
3. Fasano et al., (2005) Nat Clin Pract Gastroenterol Hepatol 2:416-422.
4. Wapenaar et al., (2008) Gut 57:463-467.
5. Rescigno et al., (2008) Curr Opin Immunol 20:669-675.
6. Fasano A (2000) Ann N Y Acad Sci 915:214-222.
7. Wang et al., (2000) J Cell Sci 113 Pt 24:4435-4440.
8. El et al., (2002) Gastroenterology 123:1607-1615.
9. Drago et al., (2006) Scand J Gastroenterol 41:408-419.
10. Fasano et al., (2000) Lancet 355:1518-1519.
11. Sapone et al., (2006) Diabetes 55:1443-1449.
12. Bowman B H, Kurosky A (1982) Adv Hum Genet 12:189-4.
13. Wicher K B, Fries E (2004) Proc Natl Acad Sci USA 101:14390-14395
14. Meddings J B, Swain M G (2000) Gastroenterology 119:1019-1028
15. Sollid L M (2002) Nat Rev Immunol 2:647-655.
16. Barone et al., (2007) Gut 56:480-488.
17. Cenac et al., (2004) J Physiol 558:913-925.
18. van der Merwe et al., (2008) Am J Physiol Gastrointest Liver Physiol 294:G441-G451.
19. Haugen et al., (1981) J Biol Chem 256:1055-1057.
20. Maeda et al., (1984) Nature 309:131-135.
21. Kurosky et al., (1980) Proc Natl Acad Sci USA 77:3388-3392.
22. Nielsen et al., (2007) J Biol Chem 282:1072-1079.
23. Polticelli et al., (2008) FEBS J 275:5648-5656.
24. Wicher K B, Fries E (2006) Proc Natl Acad Sci USA 103:4168-4173.
25. Asleh et al., (2003) Circ Res 92:1193-1200.
26. Hunt et al., (1972) Biochem Biophys Res Commun 47:699-704.
27. Hollande et al., (2001) Am J Physiol Gastro. Liver Phys. 280:G910-G921
28. Jin et al., (2002) Invest Ophthalmol Vis Sci 43:2782-2790.
29. Lammers et al., (2008) Gastroenterology 135:194-204.
30. Playford et al., (1996) Gut 39:262-266.
31. Barnard J A, McHugh K M (2006) Growth Factors in the Gastrointestinal Tract. 1 ed. in physiology of the Gastrointestinal Tract Edition IV, eds Johnson L R, Elsevier Academic Press, 2006:183-246.
32. Jacob et al., (2005) J Biol Chem 280:31936-31948.
33. Bueno L, Fioramonti J (2008) Neurogastroenterol Motil 20:580-587.
34. Raimondi et al., (2008) Am J Physiol Gastro. Liver Phys. 294:G906-G913
35. Papp (2008) Clin Chem 54:697-704.
36. Misumi et al., (1983) Biochem Biophys Res Commun 114:729-736.
37. Pavon et al., (2006) Proteomics 6 Suppl 1:S282-S292.
38. Liu et al., (2007) Biochem Biophys Res Commun 357:531-536
39. Yaish et al., (1988) Science 242:933-935.

What is claimed is:

1. A method for diagnosing an immune-mediated disease, comprising the step of:
performing a single step genotype amplification of a haptoglobin gene in a biological sample and a healthy control sample, wherein an increase in copies of a haptoglobin 2 genotype in the biological sample compared to the healthy control sample correlates to a diagnosis and severity of the immune-mediated disease in a subject.

2. The method of claim 1, wherein the single step amplification is performed using specific primers in exon 2 and exon 5 of haptoglobulin 1 (HP1) that correspond to exons 2 and 7 of haptoglobulin 2 (HP2).

3. The method of claim 2, wherein the primer sequences are shown in SEQ ID NO: 3 and SEQ ID NO: 4.

4. The method of claim 1, wherein a monozygous genotype for haptoglobin 1 (HP1-1) is indicative of zero copies of zonulin gene and correlates to no disease.

5. The method of claim 1, wherein a heterozygous genotype for haptoglobin 2 (HP1-2) is indicative of one copy of zonulin gene and correlates to a diagnosis of the immune-mediated disease.

6. The method of claim 1, wherein a homozygous genotype for haptoglobin 2 (HP2-2) is indicative of two copies of zonulin gene and correlates to a more severe disease than diagnosed for HP2-1.

7. The method of claim 1, wherein the immune-mediated disease is type 1 diabetes, systemic lupus erythematosus, celiac disease, ankylosing spondylitis, multiple sclerosis, rheumatoid arthritis, Crohn's disease, chronic kidney disease, or schizophrenia.

8. The method of claim 1, wherein the biological sample and the control sample are blood serum, urine, stool, or a tissue biopsy.

9. A method for diagnosing celiac disease, comprising:
amplifying a haptoglobin gene using specific primers in exon 2 and exon 5 of haptoglobulin 1 (HP1) from a biological sample and a healthy control sample to determine genotype, wherein an increase in copies of a haptoglobin 2 genotype in the biological sample compared to the healthy control sample correlates to a diagnosis and severity of the immune-mediated disease in a subject.

10. The method of claim 9, wherein the specific primers in exon 2 and exon 5 of haptoglobulin 1 (HP1) correspond to exons 2 and 7 of haptoglobulin 2 (HP2).

11. The method of claim 10, wherein the primer sequences are shown in SEQ ID NO: 3 and SEQ ID NO: 4.

12. The method of claim 9, wherein a monozygous genotype for haptoglobin 1 (HP1-1) is indicative of zero copies of zonulin gene and correlates to no disease.

13. The method of claim 9, wherein a heterozygous genotype for haptoglobin 2 (HP1-2) is indicative of one copy of zonulin gene and correlates to a diagnosis of the immune-mediated disease.

14. The method of claim 9, wherein a homozygous genotype for haptoglobin 2 (HP2-2) is indicative of two copies of zonulin gene and correlates to a more severe disease than diagnosed for HP2-1.

15. The method of claim 9, wherein the biological sample and the control sample are blood serum, urine, stool, or a tissue biopsy.

16. A single step method for determining the severity of an immune-mediated disease in a subject, comprising:
identifying a genotype for haptoglobulin 2 (HP2) from an amplified haptoglobin gene in a biological sample from the subject, wherein a homozygous genotype for haptoglobin 2 is indicative of two copies of zonulin gene and correlates to a more severe disease than a heterozygous genotype that is indicative of one copy of zonulin.

17. The single step method of claim 16, wherein the haptoglobin gene is amplified using specific primers in exon 2 and exon 5 of haptoglobulin 1 (HP1) that correspond to exons 2 and 7 of haptoglobulin 2 (HP2).

18. The single step method of claim 17, wherein the primer sequences are shown in SEQ ID NO: 3 and SEQ ID NO: 4.

19. The single step method of claim 16, wherein the immune-mediated disease is type 1 diabetes, systemic lupus erythematosus, celiac disease, ankylosing spondylitis, multiple sclerosis, rheumatoid arthritis, Crohn's disease, chronic kidney disease, or schizophrenia.

20. The single step method of claim 16, wherein the biological sample is blood serum, urine, stool, or a tissue biopsy.
